(19) United States
(12) Patent Application Publication
(10) Pub. No.: US 2006/0228720 A1
(43) Pub. Date: Oct. 12, 2006

(54) Method for imaging an array of microspheres

(75) Inventors: Martin C. Kaplan, Rochester, NY (US); Krishnan Chari, Fairport, NY (US); Samuel Chen, Penfield, NY (US); Douglas L. Vizard, Durham, CT (US)

Correspondence Address: Pamela R. Crocker;Patent Legal Staff, Eastman Kodak Company, 343 State Street, Rochester, NY, 14650-2201, US

(73) Assignee: Eastman Kodak Company

(21) Appl. No.: 11/103,765

(22) Filed: Apr. 12, 2005

(51) Int. Cl. C12Q 1/68; G01N 33/53; G06K 9/00

(52) U.S. Cl. 435/6; 435/7.1; 382/128

(57) ABSTRACT

A method for imaging an array of microspheres. A light source, filter or combination of filters that isolate wavelength, or wavelength ranges, of light and a camera are used for detecting and quantifying the presence of biological probes that indicate the presence of specific chemical moieties within a biological system.

### FIELD OF THE INVENTION

The present invention relates in general to molecular biological systems and more particularly to a means to simplify the detection process for colored bead random microarrays.

### BACKGROUND OF THE INVENTION

Ever since their invention in the early 1990s (Science, 251, 767-773, 1991), high-density arrays formed by the spatially addressable synthesis of bioactive probes on a 2-dimensional solid support have greatly enhanced and simplified the process of biological research and development. The key to current microarray technology is deposition of a bioactive agent at a single spot on a microchip in a “spatially addressable” manner.

Current technologies have used various approaches to fabricate microarrays. For example, U.S. Pat. No. 5,412,087, inv. McGall et al., issued on May 2, 1995 and U.S. Pat. No. 5,489,678, inv. Fodor et al., issued Feb. 6, 1996, demonstrate the use of a photolithographic process for making peptide and DNA microarrays. The patent teaches the use of photolabile protecting groups to prepare peptide and DNA microarrays through successive cycles of deprotecting a defined spot on a 1 cm.-×1 cm chip by photolithography, then flooding the entire surface with an activated amino acid or DNA base. Repetition of this process allows construction of a peptide or DNA microarray with thousands of arbitrarily different peptides or oligonucleotide sequences at different spots on the array. This method is expensive.

An ink jet approach is being used by others (e.g., Papen et al., U.S. Pat. No. 6,079,283, issued Jun. 27, 2000, U.S. Pat. No. 6,083,762; issued, Jul. 4, 2000 and U.S. Pat. No. 6,094,966, issued Aug. 1, 2002) to fabricate spatially addressable arrays, but this technique also suffers from high manufacturing cost in addition to the relatively large spot size of 40 to 100 μm. Because the number of bioactive probes to be placed on a single chip usually runs anywhere from 1000 to 100000 probes, the spatial addressing method is intrinsically expensive regardless how the chip is manufactured.

An alternative approach to the spatially addressable method is the concept of using fluorescent dye-incorporated polymeric beads to produce biological multiplexed arrays.

U.S. Pat. No. 5,981,180, inv. Chandler et al., issued Nov. 9, 1999 discloses a method of using color coded beads in conjunction with flow cytometry to perform multiplexed biological assay. Microspheres conjugated with DNA or monoclonal antibody probes on their surfaces were dyed internally with various ratios of two distinct fluorescence dyes. Hundreds of “spectrally addressed” microspheres were allowed to react with a biological sample and the “liquid array” was analyzed by passing a single microsphere through a flow cytometry cell to decode sample information.

U.S. Pat. No. 6,023,540, inv. Walt et al., issued Feb. 8, 2000 discloses the use of fiber-optic bundles with pre-etched microwells at distal ends to assemble dye loaded microspheres. The surface of each spectrally addressed microsphere was attached with a unique bioactive agent and thousands of microspheres carrying different bioactive probes combined to form “beads array” on pre-etched microwells of fiber optical bundles. More recently, a novel optically encoded microsphere approach was accomplished by using different sized zinc sulfide-capped cadmium selenide nanocrystals incorporated into microspheres (Nature Biotech. 19, 631-635, (2001)). Given the narrow band width demonstrated by these nanocrystals, this approach significantly expands the spectral barcoding capacity in microspheres.

Even though the “spectrally addressed microsphere” approach does provide an advantage in terms of its simplicity over the old fashioned “spatially addressable” approach in microarray making, recent improvements in the art make the manufacture and use of random microarrays less difficult and less expensive.

A coating technology is described in U.S. patent application Ser. No. 2003/0170392 A1 to prepare a microarray on a substrate that need not be pre-etched with microwells or premarked in any way with sites to attract the microspheres. Using unmarked substrates, or substrates that need no pre-coating preparation, provides a huge manufacturing advantage compared to the existing technologies. Color addressable mixed beads in a dispersion can be randomly distributed on a receiving layer that has no wells or sites to attract the microspheres. Using this method, the substrate does not have to be modified even though the microspheres remain immobilized on the substrate, where the bead surfaces are exposed to facilitate easier access of the analyte to probes attached to the surfaces of the beads.

U.S. patent application Ser. No. 2003/0068609 A1 discloses a coating composition and technology for making a microarray on a substrate that does not have specific sites capable of interacting physically or chemically with the microspheres. The substrate need not be pre-etched with microwells or premarked in any way with sites to attract the microspheres. Upon coating the composition on a substrate, the microspheres become immobilized in the plane of coating and form a random pattern on the substrate.

Using unmarked substrates or substrates that need no pre-coating preparation provides a manufacturing means that is less costly and easier to prepare than those previously disclosed because the substrate does not have to be modified compared to the existing technologies. A unique composition allows color addressable mixed beads to be randomly distributed on a substrate that has no wells or sites to attract the microspheres. A method of making a random array of microspheres using enzyme digestion to expose surfaces of the microspheres is taught in U.S. patent application Ser. No. 2003/0224361 A1. Enzyme digestion can be easily controlled to expose the desired amount of microsphere and the enzyme, a protease, is readily available and economical to obtain.

A method of manufacturing and detecting colored microarrays is described in U.S. patent application Ser. No. 2004/0106114 A1. During the manufacture of the microspheres, an optical bar code is generated of the colorants associated with the microspheres and stored in a digital file. The biologically/chemically active region of a support treated with the microspheres is scanned with a high-resolution color scanner to produce a color map of the locations of the randomly dispersed set of one color of microspheres. A digital file of the color map produced is linked the digital file of the color map with the support. After the microarray is exposed to an analyte, the microarray is scanned by a monochrome scanner and a bead map of the microbeads is produced. The map is linked through the digital file to the location of the colored beads when the support was manufactured.

There is still a need in the art for improved methods of detection which will make the manufacture and use of microarrays less difficult and less expensive.

### SUMMARY OF THE INVENTION

A random or ordered array of colored beads, preferably arrayed on a substrate, is imaged using a broad spectrum light source and an imaging device, such as a color camera. The beads are treated to act as probes, which can attach to various materials, such as proteins or genetic material, in a biological sample. More than one color of bead is present, with beads of different colors treated to probe for different materials, such as proteins or genetic material. A filter, or group of filters, is used to help distinguish differently colored beads from one another by isolating light of specific wavelengths or wavelength ranges. Beads are also treated with fluorescent and/or chemiluminescent markers to indicate the presence and/or quantity of the protein or genetic material. For chemiluminescent markers, the beads are imaged during the interaction of the bead with the sample material, detecting the spatial position of the chemiluminescing beads. For fluorescent markers, the tunable light source is tuned to wavelengths that stimulate fluorescence, and an image of the beads is taken through a filter that blocks the stimulating wavelength but transmits the fluorescent emitted wavelengths. Either before or after measuring the chemiluminescence or fluorescence, various wavelengths, or wavelength ranges, of light are isolated by removing wavelengths of light by passing the light through a filter, or group of filters, and the digital camera captures an image of the beads, usually with the fluorescent filter removed, at each wavelength or wavelength range. The spectral reflectance of each bead, which is termed the “color” of the bead, is determined by imaging the beads at several wavelengths.

The presence of protein/genetic material at probes containing fluorescent/chemiluminescent signal is indicated by the spatial position of the chemiluminescent/fluorescent signal. The spectrally determined “color” of the bead identifies the type of protein/genetic material for which the bead was prepared to probe, and thus the type of protein/genetic material that has been detected. There are several advantages to use of this invention. Use of a broad spectrum light source with several filters allows imaging of the colored beads at several different wavelengths, or wavelength ranges, allowing for a more detailed spectral characterization of the beads. This results in improved identification of a bead and an improved ability to distinguish one color of bead from another. This improves the use of random arrays of beads, which are less expensive to manufacture than carefully ordered arrays. Use of non-visible wavelengths (infrared and ultraviolet) of light from the tunable light source allows for a more detailed characterization of beads than may be available from a conventional color camera.

### BRIEF DESCRIPTION OF THE DRAWINGS

The foregoing and other objects, features, and advantages of the invention will be, apparent from the following more particular description of the embodiments of the invention, as illustrated in the accompanying drawings.

The elements of the drawings are not necessarily to scale relative to each other.

FIG. 1 is a diagram of the composition of a microarray.

FIG. 2 is a diagram of a method of sequentially imaging the microarray.

FIG. 3 is a diagram of a method of simultaneously imaging the microarray.

### DETAILED DESCRIPTION OF THE INVENTION

The following is a detailed description of the preferred embodiments of the invention, reference being made to the drawings in which the same reference numerals identify the same elements of structure in each of the several figures.

The present invention teaches a method for imaging a random or ordered array of microspheres, also referred to as “beads”, immobilized in a coating on a substrate. The microspheres are desirably formed to have a mean diameter in the range of 1 to 50 microns; more preferably in the range of 3 to 30 microns and most preferably in the range of 5 to 20 microns. It is preferred that the concentration of microspheres in the coating is in the range of 100 to a million per cm², more preferably 1000 to 200,000 per cm² and most preferably 10,000 to 100,000 per cm².

Although microspheres or particles having a substantially curvilinear shape are preferred because of ease of preparation, particles of other shape such as ellipsoidal or cubic particles may also be employed. Suitable methods for preparing the particles are emulsion polymerization as described in “Emulsion Polymerization” by I. Piirma, Academic Press, New York (1982) or by limited coalescence as described by T. H. Whitesides and D. S. Ross in J. Colloid Interface Science, vol. 169, pages 48-59, (1985). The particular polymer employed to make the particles or microspheres is a water immiscible synthetic polymer that may be colored. The preferred polymer is any amorphous water immiscible polymer. Examples of polymer types that are useful are polystyrene, poly(methyl methacrylate) or poly(butyl acrylate). Copolymers such as a copolymer of styrene and butyl acrylate may also be used. Polystyrene polymers are conveniently used.

The beads are treated to act as “probes”, by the attachment of bioactive agents to the surface of chemically functionalized microspheres. This can be performed according to the published procedures in the art (Bangs Laboratories, Inc, Technote #205). Some commonly used chemical functional groups include, but are not limited to, carboxyl, amino, hydroxyl, hydrazide, amide, chloromethyl, epoxy, aldehyde, etc. Examples of bioactive agents or probes include, but are not limited to, oligonucleotides, DNA and DNA fragments, PNAs, peptides, antibodies, enzymes, proteins, and synthetic molecules having biological activities.

The beads are also treated with a colorant, or combination of colorants, which allows for the detection of beads based on their color. The formed microsphere is colored using an insoluble colorant that is a pigment or dye that is not dissolved during array coating or subsequent treatment. Suitable dyes may be oil-soluble in nature. It is preferred that the dyes are non-fluorescent when incorporated in the microspheres. Methods for coating beads are broadly described by Edward Cohen and Edgar B. Gutoff in Chapter 1 of “Modern Coating And Drying Technology”, (Interfacial Engineering Series; v.1), (1992), VCH Publishers Inc., New York, N.Y. For a single layer format, suitable coating methods may include dip coating, rod coating, knife coating, blade coating, air knife coating, gravure coating, forward and reverse roll coating, and slot and extrusion coating. Beads are also treated with fluorescent and/or chemiluminescent markers to indicate the presence and/or quantity of the protein or genetic material. The location of the fluorescent and/or chemiluminescent markers are matched with the location of the colored beads to identify the probes that interacted with the biological material.

The microarray consists of two or more types of beads, each of which is treated to react with a specific moiety and has a unique color. The distribution or pattern of the microspheres on the substrate is either arrayed or entirely random. The microspheres are not attracted or held to sites that are pre-marked or predetermined .on the substrate. The term “random distribution”, as used herein, means a spatial distribution of elements showing no preference or bias. Randomness can be measured in terms of compliance with that which is expected by a Poisson distribution. The surface of the microspheres bear capture agents, or probes, which are readily accessible to analytes with which they come in contact.

During, or after, exposure to a biological sample, a random or ordered array of colored beads, preferably arrayed on a substrate, is imaged by illuminating the microarray 20 using a broad-spectrum light source 10 and an imaging device 15, such as a color camera as illustrated in FIG. 2. When chemiluminescent markers are used, the beads 25 are imaged during the interaction of the bead with the sample material, allowing the spatial position of the chemiluminescing beads to be determined. When fluorescent markers are used, the light from the light source is passed through a filter 13, or filters, to isolate wavelengths that stimulate fluorescence, and an image of the beads is taken through a filter that blocks the stimulating wavelength but transmits the fluorescent emitted wavelengths. Either before or after measuring the chemiluminescence or fluorescence, the light from the light source is passed through a filter 13, or filters, to isolate a desired wavelength, or wavelength range, and an image of the beads is collected, with the fluorescent filter removed. The filter 13, or filters, are changed to isolate a new wavelength, or wavelength range, and an image is collected at each selected wavelength, or wavelength range. The spectral reflectance of each bead, which is termed the “color” of the bead, is determined by imaging the beads at several wavelengths. A beam splitter can separate light into multiple beams, where each of the split beams of light is directed towards a sensor which has a filter in front of the sensor. Use of different filters before each sensor allows simultaneous imaging of the beads. Filters can also be used sequentially using a single sensor with more than one filter being used. Each filter is placed in the path of the light directed to the sensor and an image is obtained. A new filter is placed in the path of the light and a new image is obtained.

The presence of biological material at probes containing a fluorescent/chemiluminescent signal is indicated by the spatial position of the chemiluminescent/fluorescent signal. The spectrally determined “color” of the bead at the location of the chemiluminescent/fluorescent signal identifies the bead and the corresponding moiety for which the bead was prepared to probe.

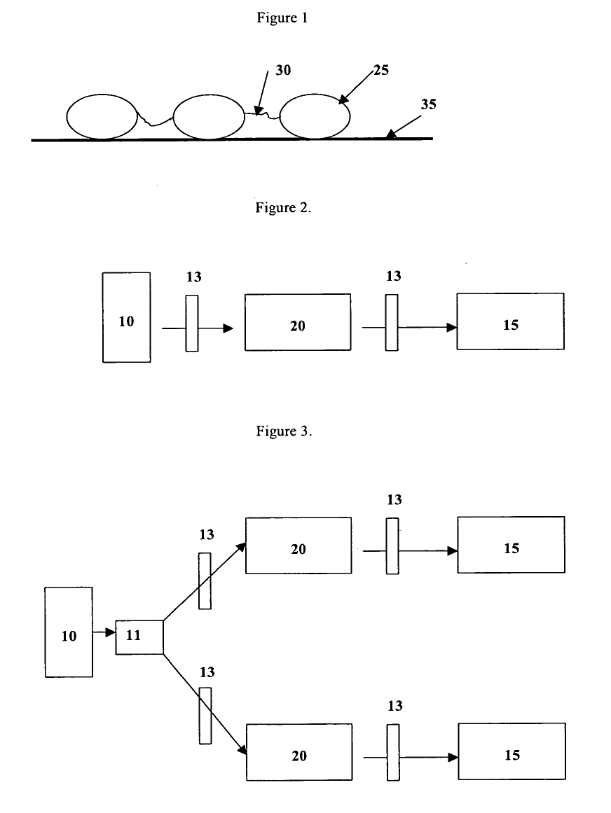

FIG. 1 shows a diagram of a microarray in accordance with the present invention. The microarray 20 is composed of colored beads 25, or microspheres, dispersed preferably ina coating 30 on a substrate 35. The beads 25 contain a biological/chemical probe 40 and at least one colorant 45.

FIG. 2 shows a diagram of a method of sequentially imaging the microarray 20 by illuminating the microarray 20 using a light source 10 and an imaging device 15, such as a color camera. A filter, or series of filters 13 is placed between the light source 10 and the microarray 20, or between the microarray 20 and the imaging device 15. Alternatively, filters 13 can be placed both between the light source 10 and the microarray 20, and between the microarray 20 and the imaging device 15. Depending upon the nature of the beads used, imaging may occur during, or after, exposure to a biological sample.

FIG. 3 shows a diagram of a method of simultaneously imaging the microarray 20 by illuminating the microarray 20 using a light source 10 and an imaging device 15, such as a color camera. Light is split into at least two beams using a beam splitter 11. The beam splitter 11 is placed between the light source 10 and the microarray 20, or between the microarray 20 and the imaging device 15. A filter, or series of filters 13 is placed in the beam of light between the beam splitter 11 and the imaging device 15. Depending upon the nature of the beads used, imaging may occur during, or after, exposure to a biological sample.

All documents, patents, journal articles and other materials cited in the present application are hereby incorporated by reference.

The invention has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

### PARTS LIST

10 light source
11 beam splitter
13 filter(s)
15 imaging device
20 microarray
25 colored beads, or microspheres
30 coating
35 substrate
40 biological/chemical probe
45 colorant

1. A method of imaging an array of colored beads, the method comprising the steps of:
exposing an array of color coded beads containing a multiple of colors, where each bead contains a single color, which is coded to identify a probe for detecting a specific chemical moiety, a specific biochemical moiety, or a combination thereof, and at least one of the colored beads contains a marker indicating the presence of biological material, to a broad-spectrum light source;
filtering the light with a filter or combination of filters, where each filter used in the combination has a different spectral response; and
capturing an image of the beads with each filter or filter combination used.

2. The method according to claim 1 wherein the color of a bead is coded to identify a probe for detecting a specific biochemical moiety.

3. The method according to claim 2 wherein the biochemical moiety is a protein.

4. The method according to claim 2 wherein the biochemical moiety is genetic material.

5. The method according to claim 1 wherein the array is spatially random.

6. The method according to claim 1 wherein the array is spatially ordered.

7. The method according to claim 1 wherein the array is supported on a substrate.

8. The method according to claim 1 wherein more than one filter, or filter combination, is used and an image is captured for each filter, or filter combination.

9. The method according to claim 1 wherein the image is captured using a camera or device with at least two identical sensors, with different filters placed between the sample and each sensor.

10. The method according to claim 1 wherein the image is captured using a camera or device with at least two different sensors, with each sensor having a different spectral response.

11. The method according to claim 1 wherein at least one color of bead is treated to act as a probe for identifying the moiety recognized by said probe, and said at least one color of bead contains a fluorescent or chemiluminescent marker to indicate the presence, quantity, or combination thereof for a chemical moiety in a biological sample.

12. The method according to claim 10 wherein at least one color of bead contains at least one fluorescent marker and at least one color of bead contain at least one chemiluminescent marker.

13. The method according to claim 1 wherein at least one color of bead is treated to act as a probe for identifying the moiety recognized by said probe, and said at least one color of bead contains a fluorescent or chemiluminescent marker to indicate the presence, quantity, or combination thereof for a protein, genetic material or other material of biological origin.

14. The method according to claim 12 wherein at least one color of bead contains at least one fluorescent marker and at least one color of bead contain at least one chemiluminescent marker.

15. The method according to claim 1 wherein at least one color of bead contains a chemiluminescent marker, and the bead is imaged during the interaction of the bead with the biological material, thereby detecting the spatial position of the chemiluminescing bead.

16. The method according to claim 1 wherein at least one color of bead contains a fluorescent marker;
the light source emits fight of wavelengths that stimulates the fluorescence; and
the bead is imaged through a filter that blocks the stimulating wavelength but transmits the fluorescent emitted wavelengths.

17. The method according to claim 1 wherein the light source and filters allow imaging at non-visible wavelengths.

18. The method according to claim 1 wherein said filters are placed between the light source and the array of colored beads.

19. The method according to claim 1 wherein said filters are placed between the array of colored beads and the device that images the beads.

20. The method according to claim 1 wherein said filters are placed between the light source and the array of colored beads and between the array of colored beads and the device that images the beads.

21. The method according to claim 1 wherein said filters are integrated into the camera sensor.

22. The method according to claim 1 wherein a single filter has an adjustable spectral response.
